(19) United States
(12) Patent Application Publication
(10) Pub. No.: US 2021/0137917 A1
(43) Pub. Date: May 13, 2021

(54) PHARMACEUTICAL COMPOSITION OF NINTEDANIB ESYLATE

(71) Applicant: INTAS PHARMACEUTICALS LTD., Ahmendabad, Gujarat (IN)

(72) Inventors: Nilesh Bhagwat TELI, Ahmedabad (IN); Pradosh Ranjan SAHOO, Ahmedabad (IN); Sunil Shantwan BORUDE, Ahmedabad (IN); Venkataramana NAIDU, Ahmedabad (IN)

(21) Appl. No.: 17/045,895

(22) PCT Filed: Apr. 8, 2019
(86) PCT No.: PCT/IB2019/052860

§ 371 Date: Oct. 7, 2020

(30) Foreign Application Priority Data
Apr. 9, 2018 (IN) 201821013537

(51) Int. Cl. A61K 31/496; A61K 9/48; A61K 47/14

(52) U.S. Cl. CPC A61K 31/496; A61K 47/14; A61K 9/4825

(57) ABSTRACT

The present invention provides a pharmaceutical composition comprising suspension of Nintedanib esylate and lauroyl polyoxyl-6 glyceride or hydrogenated vegetable oil, in a soft gelatin capsule; wherein lauroyl polyoxyl-6 glyceride or hydrogenated vegetable oil are used as a thickener. Further, the present invention provides a process for preparation of pharmaceutical composition comprising suspension of Nintedanib esylate and lauroyl polyoxyl-6 glyceride or hydrogenated vegetable oil, in a soft gelatin capsule.

### RELATED APPLICATIONS

This application is related to Indian Provisional Application IN201821013537 filed 9 Apr. 2018 and is incorporated herein in its entirety.

### FIELD OF THE INVENTION

The present invention relates to pharmaceutical composition comprising a suspension of Nintedanib esylate and lauroyl polyoxyl-6 glyceride or hydrogenated vegetable oil, in a soft gelatin capsule; and process for the preparation of the said composition.

### BACKGROUND OF THE INVENTION

Nintedanib is a small molecule that inhibits multiple receptor tyrosine kinases (RTKs), and non-receptor tyrosine kinases (nRTKs). Nintedanib inhibits the following RTKs: platelet-derived growth factor receptor (PDGFR) α, and β, fibroblast growth factor receptor (FGFR) 1-3, vascular endothelial growth factor receptor (VEGFR) 1-3, and Fms like tyrosine kinase 3 (FLT3), and nRTKs: Lck, Lyn, and Src kinases. Nintedanib is approved as anti-fibrotic, and anti-inflammatory agent. The chemical name of Nintedanib is 1H-Indole-6-carboxylic acid,2,3dihydro-3-[[[4-[methyl [(4-methyl-1-piperazinyl)acetyl]amino]phenyl] amino]phenylmethylene]-2-oxo-,methylester, (3Z). The molecular formula is C₃₁H₃₃N₅O₄, and the molecular weight is 539.636, and has following chemical structure:

[chemical structure]

U.S. Pat. No. 6,762,180 discloses substituted indolines which covers Nintedanib. Nintedanib is marketed as soft gelatin capsule (OFEV®, VARGATEF®, and CYENDIV®) by Boehringer Ingelheim.

OFEV® is approved for the treatment of idiopathic pulmonary fibrosis (IPF), and VARGATEF® is approved for combination with docetaxel for the treatment of adult patients with locally advanced, metastatic or locally recurrent non-small cell lung cancer (NSCLC) of adenocarcinoma tumour histology after first-line chemotherapy. OFEV® and VARGATEF®, are both soft gelatin capsules and contain medium chain triglycerides, hard fat, and lecithin as a part of filling materials, and gelatin, glycerol, titanium dioxide, red ferric oxide, yellow ferric oxide as a part of capsule shell. Lauroyl polyoxyl-6 glyceride or hydrogenated vegetable oil is not used in currently marketed composition.

From literature it is known that Nintedanib has poor solubility and stability at neutral conditions, hence it cannot be formulated as a solution. To overcome the problem of solubility and stability, several approaches are reported as follows:

U.S. Pat. No. 8,143,247 discloses hard or soft gelatin capsule comprising Nintedanib, medium chain triglycerides in the form of aqueous suspension or oil-in-water emulsion. The composition further comprises dispersing or wetting agents which may be a naturally occurring phosphatide such as lecithin.

US20160324791 discloses composition comprising Nintedanib in lipid suspension, medium chain triglycerides as a lipid carrier, hard fat as a thickener, and lecithin as a glidant/solubilizing agent.

CN105963268 discloses composition comprising Nintedanib in the form of dispersible tablet.

Considering the prior efforts as disclosed in the background, still a need exists which would address the issues relating to solubility and stability of Nintedanib esylate in the pharmaceutical composition.

### OBJECT OF THE INVENTION

It is therefore, an object of the present invention is to provide pharmaceutical composition comprising Nintedanib esylate and lauroyl polyoxyl-6 glyceride.

Another object of the present invention is to provide process for the preparation of pharmaceutical composition comprising Nintedanib esylate and lauroyl polyoxyl-6 glyceride.

Another object of the present invention is to provide pharmaceutical composition comprising suspension of Nintedanib esylate and lauroyl polyoxyl-6 glyceride.

Another object of the present invention is to provide pharmaceutical composition comprising suspension of Nintedanib esylate, lauroyl polyoxyl-6 glyceride as a thickener, a carrier, and a glidant/solubilizing agent.

Another object of the present invention is to provide pharmaceutical composition comprising suspension of Nintedanib esylate, lauroyl polyoxyl-6 glyceride as a thickener, medium chain triglyceride as a carrier, and lecithin as a glidant/solubilizing agent.

Another object of the present invention is to provide pharmaceutical composition comprising suspension of Nintedanib esylate, lauroyl polyoxyl-6 glyceride as a thickener, medium chain triglyceride as a carrier, and lecithin as a glidant/solubilizing agent in a capsule.

Another object of the present invention is to provide a capsule comprising a capsule shell and a capsule composition, characterized in that the capsule composition comprises the suspension of Nintedanib esylate, lauroyl polyoxyl-6 glyceride as a thickener, medium chain triglyceride as a carrier, and lecithin as a glidant/solubilizing agent.

Another object of the present invention is to provide a capsule comprising a capsule shell and a capsule composition, characterized in that the capsule composition comprises the suspension of Nintedanib esylate, lauroyl polyoxyl-6 glyceride as a thickener, medium chain triglyceride as a carrier, and lecithin as a glidant/solubilizing agent; wherein the said capsule is a soft gelatin capsule.

Another object of the present invention is to provide pharmaceutical composition comprising Nintedanib esylate and hydrogenated vegetable oil.

Another object of the present invention is to provide process for the preparation of pharmaceutical composition comprising Nintedanib esylate and hydrogenated vegetable oil.

Another object of the present invention is to provide pharmaceutical composition comprising suspension of Nintedanib esylate and hydrogenated vegetable oil.

Another object of the present invention is to provide pharmaceutical composition comprising suspension of Nintedanib esylate, hydrogenated vegetable oil as a thickener, a carrier, and a glidant/solubilizing agent.

Another object of the present invention is to provide pharmaceutical composition comprising suspension of Nintedanib esylate, hydrogenated vegetable oil as a thickener, medium chain triglyceride as a carrier, and lecithin as a glidant/solubilizing agent.

Another object of the present invention is to provide pharmaceutical composition comprising suspension of Nintedanib esylate, hydrogenated vegetable oil as a thickener, medium chain triglyceride as a carrier, and lecithin as a glidant/solubilizing agent in a capsule.

Another object of the present invention is to provide a capsule comprising a capsule shell, and a capsule composition, characterized in that the capsule composition comprises the suspension of Nintedanib esylate, hydrogenated vegetable oil as a thickener, medium chain triglyceride as a carrier, and lecithin as a glidant/solubilizing agent.

Another object of the present invention is to provide a capsule comprising a capsule shell, and a capsule composition, characterized in that the capsule composition comprises the suspension of Nintedanib esylate, hydrogenated vegetable oil as a thickener, medium chain triglyceride as a carrier, and lecithin as a glidant/solubilizing agent; wherein the said capsule is a soft gelatin capsule.

Another object of the present invention is to provide a stable pharmaceutical composition comprising Nintedanib esylate and lauroyl polyoxyl-6 glyceride, wherein the total impurity in the said pharmaceutical composition is not more than 2% w/w of Nintedanib esylate after subjecting the said pharmaceutical composition to a stability study at 40° C./75% RH for 1 month.

Another object of the present invention is to provide a stable pharmaceutical composition comprising Nintedanib esylate and lauroyl polyoxyl-6 glyceride, wherein assay of Nintedanib esylate is 90% to 110% after subjecting the said pharmaceutical composition to a stability study at 40° C./75% RH for 1 month.

Another object of the present invention is to provide a stable pharmaceutical composition comprising Nintedanib esylate and lauroyl polyoxyl-6 glyceride, wherein the total impurity in the said pharmaceutical composition is not more than 2% w/w of Nintedanib esylate after subjecting the said pharmaceutical composition to a stability study at 40° C./75% RH for 6 months.

Another object of the present invention is to provide a stable pharmaceutical composition comprising Nintedanib esylate and lauroyl polyoxyl-6 glyceride, wherein assay of Nintedanib esylate is 90% to 110% after subjecting the said pharmaceutical composition to a stability study at 40° C./75% RH for 6 months.

Another object of the present invention is to provide a soft gelatin capsule comprising a suspension composition comprising: (a) Nintedanib esylate; (b) 5-25% w/w lauroyl polyoxyl-6 glyceride of the total suspension composition; and (c) one or more pharmaceutically acceptable excipients; wherein, the soft gelatin capsule exhibits a dissolution profile according to which: (1) up to 75 wt % of Nintedanib esylate is dissolved in 10 minutes; and (2) more than 75 wt % Nintedanib esylate is dissolved in 60 minutes when dissolution study is performed using 900 mL 0.1 N HCl as dissolution medium at 37° C. and 100 rpm in USP apparatus type II; and wherein, total impurity in the soft gelatin capsule is not more than 2% w/w of Nintedanib esylate after stability study at 40° C. and 75% RH for 6 months.

Another object of the present invention is to provide a stable pharmaceutical composition comprising Nintedanib esylate and hydrogenated vegetable oil, wherein the total impurity in the said pharmaceutical composition is not more than 2% w/w of Nintedanib esylate after subjecting the said pharmaceutical composition to a stability study at 40° C./75% RH for 1 month.

Another object of the present invention is to provide a stable pharmaceutical composition comprising Nintedanib esylate and hydrogenated vegetable oil, wherein assay of Nintedanib esylate is 90% to 110% after subjecting the said pharmaceutical composition to a stability study at 40° C./75% RH for 1 month.

Another object of the present invention is to provide a stable pharmaceutical composition comprising Nintedanib esylate and hydrogenated vegetable oil, wherein the total impurity in the said pharmaceutical composition is not more than 2% w/w of Nintedanib esylate after subjecting the said pharmaceutical composition to a stability study at 40° C./75% RH for 6 months.

Another object of the present invention is to provide a stable pharmaceutical composition comprising Nintedanib esylate and hydrogenated vegetable oil, wherein assay of Nintedanib esylate is 90% to 110% after subjecting the said pharmaceutical composition to a stability study at 40° C./75% RH for 6 months.

Another object of the present invention is to provide a soft gelatin capsule comprising a suspension composition comprising: (a) Nintedanib esylate; (b) 1-10% w/w hydrogenated vegetable oil of the total suspension composition; and (c) one or more pharmaceutically acceptable excipients; wherein, the soft gelatin capsule exhibits a dissolution profile according to which: (1) up to 75 wt % of Nintedanib esylate is dissolved in 10 minutes; and (2) more than 75 wt % Nintedanib esylate is dissolved in 60 minutes when dissolution study is performed using 900 mL 0.1 N HCl as dissolution medium at 37° C. and 100 rpm in USP apparatus type II; and wherein, total impurity in the soft gelatin capsule is not more than 2% w/w of Nintedanib esylate after stability study at 40° C. and 75% RH for 6 months.

### SUMMARY OF THE INVENTION

The present invention provides a pharmaceutical composition comprising suspension of Nintedanib esylate and lauroyl polyoxyl-6 glyceride or hydrogenated vegetable oil, in a soft gelatin capsule; wherein lauroyl polyoxyl-6 glyceride or hydrogenated vegetable oil are used as a thickener. Further, the present invention provides a process for preparation of pharmaceutical composition comprising suspension of Nintedanib esylate and lauroyl polyoxyl-6 glyceride or hydrogenated vegetable oil, in a soft gelatin capsule.

### DETAILED DESCRIPTION OF THE INVENTION

The present invention provides a pharmaceutical composition comprising Nintedanib esylate and lauroyl polyoxyl-6 glyceride.

In another embodiment the present invention provides a process for the preparation of pharmaceutical composition comprising Nintedanib esylate and lauroyl polyoxyl-6 glyceride.

In another embodiment, the present invention provides a pharmaceutical composition comprising Nintedanib esylate and lauroyl polyoxyl-6 glyceride, which further comprises of suitable excipients.

In another embodiment the present invention provides a pharmaceutical composition comprising suspension of Nintedanib esylate and lauroyl polyoxyl-6 glyceride.

In another embodiment the present invention provides a pharmaceutical composition comprising suspension of Nintedanib esylate, lauroyl polyoxyl-6 glyceride as a thickener, a carrier, and a glidant/solubilizing agent.

In another embodiment the present invention provides a pharmaceutical composition comprising suspension of Nintedanib esylate, lauroyl polyoxyl-6 glyceride as a thickener, medium chain triglyceride as a carrier, and lecithin as a glidant/solubilizing agent.

In another embodiment the present invention provides a pharmaceutical composition comprising suspension of Nintedanib esylate, lauroyl polyoxyl-6 glyceride as a thickener, medium chain triglyceride as a carrier, and lecithin as a glidant/solubilizing agent in a capsule.

In another embodiment the present invention provides a capsule comprising a capsule shell and a capsule composition, characterized in that the capsule composition comprises the suspension of Nintedanib esylate, lauroyl polyoxyl-6 glyceride as a thickener, medium chain triglyceride as a carrier, and lecithin as a glidant/solubilizing agent.

In another embodiment the present invention provides a capsule comprising a capsule shell and a capsule composition, characterized in that the capsule composition comprises the suspension of Nintedanib esylate, lauroyl polyoxyl-6 glyceride as a thickener, medium chain triglyceride as a carrier, and lecithin as a glidant/solubilizing agent; wherein the said capsule is a soft gelatin capsule.

In another embodiment the present invention provides a pharmaceutical composition comprising Nintedanib esylate and hydrogenated vegetable oil.

In another embodiment the present invention provides a process for the preparation of pharmaceutical composition comprising Nintedanib esylate and hydrogenated vegetable oil.

In another embodiment, the present invention provides a pharmaceutical composition comprising Nintedanib esylate and hydrogenated vegetable oil, which further comprises of suitable excipients.

In another embodiment the present invention provides a pharmaceutical composition comprising suspension of Nintedanib esylate and hydrogenated vegetable oil.

In another embodiment the present invention provides a pharmaceutical composition comprising suspension of Nintedanib esylate, hydrogenated vegetable oil as a thickener, a carrier, and a glidant/solubilizing agent.

In another embodiment the present invention provides a pharmaceutical composition comprising suspension of Nintedanib esylate, hydrogenated vegetable oil as a thickener, medium chain triglyceride as a carrier, and lecithin as a glidant/solubilizing agent.

In another embodiment the present invention provides a pharmaceutical composition comprising suspension of Nintedanib esylate, hydrogenated vegetable oil as a thickener, medium chain triglyceride as a carrier, and lecithin as a glidant/solubilizing agent in a capsule.

In another embodiment the present invention provides a capsule comprising a capsule shell, and a capsule composition, characterized in that the capsule composition comprises the suspension of Nintedanib esylate, hydrogenated vegetable oil as a thickener, medium chain triglyceride as a carrier, and lecithin as a glidant/solubilizing agent.

In another embodiment the present invention provides a capsule comprising a capsule shell, and a capsule composition, characterized in that the capsule composition comprises the suspension of Nintedanib esylate, hydrogenated vegetable oil as a thickener, medium chain triglyceride as a carrier, and lecithin as a glidant/solubilizing agent; wherein the said capsule is a soft gelatin capsule.

Lauroyl polyoxyl-6 glyceride is currently marketed and available as LABRAFIL® M 2130 CS by Gattefossé, which is designed for use in pharmaceutical compositions administered by oral, topical and/or rectal/vaginal routes. LABRAFIL® M 2130 CS is a waxy solid and easily soluble in chloroform, and methylene chloride. LABRAFIL® M 2130 CS has melting point range between 33-38° C. LABRAFIL® M 2130 CS is a suitable thickener as compared to hard fat, it stabilizes the suspension system, ensures optimal processing and guarantees an adequate capsule quality; especially as far as content uniformity or dissolution behavior is concerned.

Hydrogenated vegetable oil is currently marketed and available as BBS-C® by Abitec Corporation, which is designed for use in suspension compositions in soft gels, and syrups. BBS-C® is a partially hydrogenated vegetable oil (soybean, and cottonseed) with bland flavor, good stability, superior creaming, and resistance to oxidation. BBS-C® has melting point range between 20-50° C.

According to present invention suitable excipients may include, but not limited to carrier, thickener, glidant/solubilizing agent, and likes thereof.

According to present invention, carrier may include, but not limited to corn oil glycerides, medium chain triglycerides, medium chain partial glycerides, oleic acid, sorbitan monostearate, or mixtures thereof.

According to present invention, thickener may include hydrogenated vegetable oil, partially hydrogenated vegetable oil, lauroyl polyoxyl-6 glyceride, or mixtures thereof.

According to present invention, solubilizing agent may include, but not limited to, lecithin, polysorbate, hydroxypropyl betadex, macrogol, polyoxyethylene alkyl ethers, or mixtures thereof.

According to present invention, glidant may include, but not limited to calcium silicate, lecithin, magnesium silicate, colloidal silicon dioxide, talc, or mixtures thereof.

In another embodiment, the present invention provides a soft gelatin capsule comprising a suspension of Nintedanib esylate, medium chain triglyceride, lauroyl polyoxyl-6 glyceride, and lecithin.

In another embodiment, the present invention provides a soft gelatin capsule comprising a suspension of Nintedanib esylate, medium chain triglyceride, hydrogenated vegetable oil, and lecithin.

According to present invention, Nintedanib esylate can be present in an amount ranging from about 30% to about 60%, weight by weight of the total suspension composition.

According to present invention, medium chain triglyceride can be present in an amount ranging from about 35% to about 65%, weight by weight of the total suspension composition.

According to present invention, lauroyl polyoxyl-6 glyceride can be present in an amount ranging from about 5% to about 25%, weight by weight of the total suspension composition.

According to present invention, hydrogenated vegetable oil can be present in an amount ranging from about 1% to about 10%, weight by weight of the total suspension composition.

According to present invention, lecithin can be present in an amount ranging from about 0.1% to about 5%, weight by weight of the total suspension composition.

In another embodiment the present invention provides a process for the preparation of pharmaceutical composition comprising Nintedanib esylate and lauroyl polyoxyl-6 glyceride or hydrogenated vegetable oil comprising step of:

(1) mixing Lauroyl polyoxyl-6 glycerides or hydrogenated vegetable oil, and part of medium chain triglycerides,

(2) mixing lecithin, rest of the part of medium chain triglycerides and the Nintedanib esylate to obtain a suspension,

(3) mixing, homogenizing, deaerating, and sieving the suspension of step 2 to produce final suspension composition, and

(4) encapsulating the final suspension composition of step 3 to obtain a soft gelatin capsule.

In another embodiment, the present invention provides a soft gelatin capsule comprising capsule shell and capsule composition; wherein

(a) the capsule shell comprises one or more plasticizing agents, one or more capsule shell formers, one or more opacifiers, one or more colorants, one or more solvents, and optionally further auxiliary materials, and

(b) the capsule composition comprises Nintedanib esylate composition as hereinbefore described.

In another embodiment, the present invention provides a stable pharmaceutical composition comprising Nintedanib esylate and lauroyl polyoxyl-6 glyceride, wherein the total impurity in the said pharmaceutical composition is not more than 2% w/w of Nintedanib esylate after subjecting the said pharmaceutical composition to a stability study at 40° C./75% RH for 1 month.

In another embodiment, the present invention provides a stable pharmaceutical composition comprising Nintedanib esylate and lauroyl polyoxyl-6 glyceride, wherein assay of Nintedanib esylate is 90% to 110% after subjecting the said pharmaceutical composition to a stability study at 40° C./75% RH for 1 month.

In another embodiment, the present invention provides a stable pharmaceutical composition comprising Nintedanib esylate and lauroyl polyoxyl-6 glyceride, wherein the total impurity in the said pharmaceutical composition is not more than 2% w/w of Nintedanib esylate after subjecting the said pharmaceutical composition to a stability study at 40° C./75% RH for 6 months.

In another embodiment, the present invention provides a stable pharmaceutical composition comprising Nintedanib esylate and lauroyl polyoxyl-6 glyceride, wherein assay of Nintedanib esylate is 90% to 110% after subjecting the said pharmaceutical composition to a stability study at 40° C./75% RH for 6 months.

In another embodiment, the present invention provides a soft gelatin capsule comprising a suspension composition comprising: (a) Nintedanib esylate; (b) 5-25% w/w lauroyl polyoxyl-6 glyceride of the total suspension composition; and (c) one or more pharmaceutically acceptable excipients; wherein, the soft gelatin capsule exhibits a dissolution profile according to which: (1) up to 75 wt % of Nintedanib esylate is dissolved in 10 minutes; and (2) more than 75 wt % Nintedanib esylate is dissolved in 60 minutes when dissolution study is performed using 900 mL 0.1 N HCl as dissolution medium at 37° C. and 100 rpm in USP apparatus type II; and wherein, total impurity in the soft gelatin capsule is not more than 2% w/w of Nintedanib esylate after stability study at 40° C. and 75% RH for 6 months.

In another embodiment, the present invention provides a stable pharmaceutical composition comprising Nintedanib esylate and hydrogenated vegetable oil, wherein the total impurity in the said pharmaceutical composition is not more than 2% w/w of Nintedanib esylate after subjecting the said pharmaceutical composition to a stability study at 40° C./75% RH for 1 month.

In another embodiment, the present invention provides a stable pharmaceutical composition comprising Nintedanib esylate and hydrogenated vegetable oil, wherein assay of Nintedanib esylate is 90% to 110% after subjecting the said pharmaceutical composition to a stability study at 40° C./75% RH for 1 month.

In another embodiment, the present invention provides a stable pharmaceutical composition comprising Nintedanib esylate and hydrogenated vegetable oil, wherein the total impurity in the said pharmaceutical composition is not more than 2% w/w of Nintedanib esylate after subjecting the said pharmaceutical composition to a stability study at 40° C./75% RH for 6 months.

In another embodiment, the present invention provides a stable pharmaceutical composition comprising Nintedanib esylate and hydrogenated vegetable oil, wherein assay of Nintedanib esylate is 90% to 110% after subjecting the said pharmaceutical composition to a stability study at 40° C./75% RH for 6 months.

In another embodiment, the present invention provides a soft gelatin capsule comprising a suspension composition comprising: (a) Nintedanib esylate; (b) 1-10% w/w hydrogenated vegetable oil of the total suspension composition; and (c) one or more pharmaceutically acceptable excipients; wherein, the soft gelatin capsule exhibits a dissolution profile according to which: (1) up to 75 wt % of Nintedanib esylate is dissolved in 10 minutes; and (2) more than 75 wt % Nintedanib esylate is dissolved in 60 minutes when dissolution study is performed using 900 mL 0.1 N HCl as dissolution medium at 37° C. and 100 rpm in USP apparatus type II; and wherein, total impurity in the soft gelatin capsule is not more than 2% w/w of Nintedanib esylate after stability study at 40° C. and 75% RH for 6 months.

### EXAMPLES

The present invention has been described by way of example only, and it is to be recognized that modifications thereto falling within the scope, and spirit of appended claims, and which would be obvious to a person skilled in the art based upon the disclosure herein, are also considered to be within the scope of this invention.

### Example: Nintedanib 100 and 150 mg Capsule

| | | Example 1 | Example 2 |
|---|---|---|---|
| Sr. No. | Ingredients | % | % |
| 1 | Nintedanib esylate | 30-60 | 30-60 |
| 2 | Medium chain triglyceride | 35-65 | 35-65 |
| 3 | Lauroyl polyoxyl-6 glycerides | 5-25 | — |
| 4 | Hydrogenated vegetable oil | — | 1-10 |
| 5 | Lecithin | 0.1-5 | 0.1-5 |
| 6 | Gelatin | 35-50 | 35-50 |
| 7 | Glycerol | 15-25 | 15-25 |
| 8 | Titanium dioxide | 0.1-3 | 0.1-3 |
| 9 | Iron oxide red | 0.01-1 | 0.01-1 |
| 10 | Iron oxide yellow | 0.01-1 | 0.01-1 |
| 11 | Purified water | 25-50 | 25-50 |

### Manufacturing Process for Example 1 and 2

1. Lauroyl polyoxyl-6 glycerides or hydrogenated vegetable oil, and part of medium chain triglycerides were mixed.
2. Subsequently lecithin, the rest of the part of medium chain triglycerides, and the Nintedanib esylate were added to obtain a suspension.
3. The suspension was properly mixed, homogenized, deaerated, and finally sieved to produce final suspension composition.
4. The final suspension composition was encapsulation to obtain a soft gelatin capsule.

### Example 3: Nintedanib 100 and 150 mg Capsule

| | | 100 mg | 150 mg |
|---|---|---|---|
| Sr. No. | Ingredients | % | % |
| 1 | Nintedanib esylate | 43.00 | 43.00 |
| 2 | Medium chain triglyceride | 44.07 | 44.07 |
| 3 | Lauroyl polyoxyl-6 glycerides | 12.50 | 12.50 |
| 4 | Lecithin | 0.43 | 0.43 |

Capsule shell

| | | | |
|---|---|---|---|
| 5 | Gelita RXL adv. 190 bloom, porcine skin | 43.00 | 43.00 |
| 6 | Glycerol 85% | 19.00 | 19.00 |
| 7 | Titanium dioxide | 0.55 | 0.14 |
| 8 | Iron oxide red | 0.02 | 0.38 |
| 9 | Iron oxide yellow | 0.03 | 0.45 |
| 10 | Purified water | 37.40 | 37.04 |

### Example 4: Nintedanib 100 and 150 mg Capsule

| | | 100 mg | 150 mg |
|---|---|---|---|
| Sr. No. | Ingredients | % | % |
| 1 | Nintedanib esylate | 43.00 | 43.00 |
| 2 | Medium chain triglyceride | 53.00 | 53.00 |
| 3 | Hydrogenated vegetable oil | 3.57 | 3.57 |
| 4 | Lecithin | 0.43 | 0.43 |

Capsule shell

| | | | |
|---|---|---|---|
| 5 | Gelita RXL adv. 190 bloom, porcine skin | 43.00 | 43.00 |
| 6 | Glycerol 85% | 19.00 | 19.00 |
| 7 | Titanium dioxide | 0.55 | 0.14 |
| 8 | Iron oxide red | 0.02 | 0.38 |
| 9 | Iron oxide yellow | 0.03 | 0.45 |
| 10 | Purified water | 37.40 | 37.04 |

Manufacturing process: Nintedanib capsule of example 3 and 4 was prepared by the similar process as of example 1 and 2.

### Example 5: Nintedanib 100 and 150 mg Capsule (mg/Capsule)

| | | 100 mg | 150 mg |
|---|---|---|---|
| Sr. No. | Ingredients | (mg) | (mg) |
| 1 | Nintedanib esylate | 120.40 | 180.60 |
| 2 | Medium chain triglyceride | 123.40 | 185.10 |
| 3 | Lauroyl polyoxyl-6 glycerides | 35.00 | 52.50 |
| 4 | Lecithin | 1.20 | 1.80 |

| | | |
|---|---|---|
| Total | 280 | 420 |

### Example 6: Nintedanib 100 and 150 mg Capsule (mg/Capsule)

| | | 100 mg | 150 mg |
|---|---|---|---|
| Sr. No. | Ingredients | (mg) | (mg) |
| 1 | Nintedanib esylate | 120.40 | 180.60 |
| 2 | Medium chain triglyceride | 148.40 | 222.60 |
| 3 | Hydrogenated vegetable oil | 10.00 | 15.00 |
| 4 | Lecithin | 1.20 | 1.80 |

| | | |
|---|---|---|
| Total | 280 | 420 |

### Manufacturing Process for Example 5 and 6

1. Lauroyl polyoxyl-6 glycerides or hydrogenated vegetable oil, and part of medium chain triglycerides were pre-mixed in the processing unit.
2. Subsequently lecithin, the rest of the part of medium chain triglycerides, and the active substance were added to obtain a suspension.
3. The suspension was mixed, homogenized, deaerated, and finally sieved to produce final suspension composition.
4. The final suspension composition is further encapsulated to obtain soft gelatin capsule.

Soft gelatin capsule obtained according to example 3 (150 mg), example 4 (150 mg) and OFEV® 150 mg—reference listed drug (RLD) for Nintedanib esylate were subjected to stability and dissolution study.

Stability/Dissolution Study Results:

The stability study of the soft gelatin capsule prepared according to example 3 (150 mg), example 4 (150 mg) and OFEV®—150 mg (RLD) were charged for stability study at accelerated and long term storage condition and chemical parameters were evaluated. The stability results obtained are tabulated below. The soft gelatin capsule prepared according to example 3 and 4 were found to be stable.

| | | OFEV ® - 150 mg (RLD) | | | Example 3 (150 mg) | | | |
|---|---|---|---|---|---|---|---|---|
| Sr. No. | Tests | | 40° C., 75% | 40° C., 75% | | 40° C., 75% | 40° C., 75% | 25° C., 60% |
| Conditions | | Initial | RH, 1 M | RH, 6 M | Initial | RH, 1 M | RH, 6 M | RH, 6 M |
| 1 | Assay (%) | 101.40 | NP | NP | 99.20 | 99.30 | 100.7 | 100.8 |
| | Related Substances | | | | | | | |
| 2 | Acid impurity | ND | ND | ND | 0.055 | 0.051 | 0.059 | 0.059 |
| | N-Oxide impurity | ND | ND | 0.007 | ND | ND | ND | ND |
| | Highest individual | 0.026 | 0.027 | 0.055 | 0.021 | 0.020 | 0.047 | 0.048 |
| | unspecified impurity | | | | | | | |
| | Total impurity | 0.09 | 0.089 | 0.093 | 0.186 | 0.204 | 0.187 | 0.185 |
| | Dissolution Profile: 0.1N HCl, Type II (Paddle with Sinker), 100 RPM, 900 ml | | | | | | | |
| | Time (Minutes) | % Release | | | | | | |
| 3 | 10 | 92 | 43 | NP | 73 | 40 | NP | NP |
| | 30 | 98 | 78 | NP | 99 | 95 | NP | NP |
| | 45 | 99 | 91 | 93 | 99 | 99 | 101 | 102 |
| | 60 | 100 | 96 | 94 | 99 | 100 | 102 | 102 |

| | | OFEV ® - 150 mg (RLD) | | | Example 4 (150 mg) | | | |
|---|---|---|---|---|---|---|---|---|
| Sr. No | Tests | | 40° C., 75% | 40° C., 75% | | 40° C., 75% | 40° C., 75% | 25° C., 60% |
| Conditions | | Initial | RH, 1 M | RH, 6 M | Initial | RH, 1 M | RH, 6 M | RH, 6 M |
| 1 | Assay (%) | 101.40 | NP | NP | 99.10 | 99.70 | 98.5 | 100.7 |
| | Related Substances | | | | | | | |
| 2 | Acid impurity | ND | ND | ND | 0.053 | 0.053 | 0.058 | 0.057 |
| | N-Oxide impurity | ND | ND | 0.007 | ND | ND | ND | ND |
| | Highest individual | 0.026 | 0.027 | 0.055 | 0.020 | 0.030 | 0.046 | 0.047 |
| | unspecified impurity | | | | | | | |
| | Total impurity | 0.09 | 0.089 | 0.093 | 0.179 | 0.149 | 0.172 | 0.187 |
| | Dissolution Profile: 0.1N HCl, Type II (Paddle with Sinker), 100 RPM, 900 ml | | | | | | | |
| | Time (Minutes) | % Release | | | | | | |
| 3 | 10 | 92 | 43 | NP | 74 | 41 | NP | NP |
| | 30 | 98 | 78 | NP | 96 | 85 | NP | NP |
| | 45 | 99 | 91 | 93 | 96 | 97 | 98 | 99 |
| | 60 | 100 | 96 | 94 | 97 | 99 | 98 | 99 |

Wherein, RH: Relative humidity, M: Month, ND: Not detected, RPM: Rotation per minutes, NP: Not Performed

The dissolution study of the soft gelatin capsule prepared according to example 3 (150 mg), example 4 (150 mg) and OFEV®—150 mg (RLD) were carried out in 900 ml 0.1 N HCl as dissolution media in USP apparatus type II (paddle) at 100 RPM at 37° C. The dissolution study results obtained are tabulated above. The rate of dissolution were similar for OFEV®—150 mg and soft gelatin capsules obtained according to example 3 and 4 even after 6 month when stored at 40 degrees and 75 RH.

In-Vivo Bioequivalence Study Results:

Bioequivalence study of the composition obtained in example 3 (150 mg) was done against the RLD OFEV®—150 mg.

A summary of pharmacokinetic parameters are presented in below table,

| | Example 3 | OFEV ® - | | 90% Confidence Interval (%) | | |
|---|---|---|---|---|---|---|
| Pharmacokinetic Parameters | (150 mg) | 150 mg (RLD) | Ratio (%)^{e} | Lower | Upper | Power |

Dosing: Single Oral Dose; Condition: Fasting; Population: Healthy Subjects

| | | | | | | |
|---|---|---|---|---|---|---|
| N | 90 | 90 | — | — | — | — |
| Tₘₐₓ (h)^{a} | 3.249 | 3.226 | — | — | — | — |
| Cₘₐₓ (ng/mL)^{b} | 34.782 | 36.807 | 94.9 | N/AP | N/AP | N/AP |
| AUC₀₋ₜ (ng · h/mL)^{c} | 332.487 | 341.624 | 96.1 | 89.42 | 103.29 | 100.0 |
| AUC_{0-inf} (ng · h/mL)^{d} | 345.903 | 356.160 | 95.9 | 89.48 | 102.82 | 100.0 |

Dosing: Single Oral Dose; Condition: Fed; Population: Healthy Subjects

| | | | | | | |
|---|---|---|---|---|---|---|
| N | 102 | 106 | — | — | — | — |
| Tₘₐₓ (h)^{a} | 4.381 | 4.143 | — | — | — | — |
| Cₘₐₓ (ng/mL)^{b} | 43.292 | 43.116 | 99.2 | 94.41 | 104.20 | 100.0 |
| AUC₀₋ₜ (ng · h/mL)^{c} | 404.374 | 399.029 | 100.4 | 97.36 | 103.61 | 100.0 |
| AUC_{0-inf} (ng · h/mL)^{d} | 417.625 | 412.948 | 100.3 | 97.26 | 103.37 | 100.0 |

N/AP—Not Applicable
N—Total no of male subjects contributing to the summary characteristics for PK parameters
^{a}Time to maximum plasma concentration (Tₘₐₓ)
^{b}Maximum plasma concentration (Cₘₐₓ)
^{c}AUC from time zero to last measurable concentration (AUC₀₋ₜ)
^{d}AUC from time zero to infinity (AUC_{0-inf})
^{e}Ratio of least square means (Example 3 (150 mg)/OFEV ® - 150 mg (RLD))

Thus, pharmaceutical composition comprising a suspension of Nintedanib esylate and lauroyl polyoxyl-6 glyceride or hydrogenated vegetable oil can be prepared which meets the solubility and stability requirements. The pharmaceutical composition according to the present invention also bioequivalent to the RLD OFEV®.

1. A soft gelatin capsule comprising a suspension composition comprising:
a) Nintedanib esylate;
b) 5-25% w/w Lauroyl polyoxyl-6 glyceride of the total suspension composition; and
c) one or more pharmaceutically acceptable excipients;
Wherein, the soft gelatin capsule exhibits a dissolution profile according to which: (1) up to 75 wt % of Nintedanib esylate is dissolved in 10 minutes; and (2) more than 75 wt % Nintedanib esylate is dissolved in 60 minutes when dissolution study is performed using 900 mL 0.1 N HCl as dissolution medium at 37° C. and 100 rpm in USP apparatus type II;
Wherein, total impurity in the soft gelatin capsule is not more than 2% w/w of Nintedanib esylate after stability study at 40° C. and 75% RH for 6 months.

2. A soft gelatin capsule comprising a suspension composition comprising:
a) Nintedanib esylate;
b) 1-10% w/w Hydrogenated vegetable oil of the total suspension composition; and
c) one or more pharmaceutically acceptable excipients;
Wherein, the soft gelatin capsule exhibits a dissolution profile according to which: (1) up to 75 wt % of Nintedanib esylate is dissolved in 10 minutes; and (2) more than 75 wt % Nintedanib esylate is dissolved in 60 minutes when dissolution study is performed using 900 mL 0.1 N HCl as dissolution medium at 37° C. and 100 rpm in USP apparatus type II;
Wherein, total impurity in the soft gelatin capsule is not more than 2% w/w of Nintedanib esylate after stability study at 40° C. and 75% RH for 6 months.

3. The soft gelatin capsule as claimed in claim 1, wherein Nintedanib esylate is present in an amount ranging from 30-60% w/w of the total suspension composition.

4. The soft gelatin capsule as claimed in claim 1, which further comprises medium chain triglyceride present in an amount ranging from 35-65% w/w of the total suspension composition.

5. The soft gelatin capsule as claimed in claim 1, which further comprises lecithin present in an amount ranging from 0.1-5% w/w of the total suspension composition.

6. The soft gelatin capsule comprises a capsule shell and a capsule composition, wherein the capsule composition comprises a composition as claimed in claim 1.

7. The soft gelatin capsule as claimed in claim 6, wherein capsule shell comprises gelatin, glycerol, titanium dioxide, red iron oxide, yellow iron oxide and purified water.

8. A process for the preparation of soft gelatin capsule comprising Nintedanib esylate and lauroyl polyoxyl-6 glyceride or hydrogenated vegetable oil comprising step of:
(a) Mixing lauroyl polyoxyl-6 glycerides or hydrogenated vegetable oil, and part of medium chain triglycerides;
(b) Mixing lecithin, rest of the part of medium chain triglycerides and the Nintedanib esylate to obtain a suspension;
(c) Mixing, homogenizing, deaerating, and sieving the suspension of step (a) and (b) to produce final suspension composition; and
(d) Encapsulating the final suspension composition of step (c) to obtain a soft gelatin capsule.

9. The soft gelatin capsule as claimed in claim 2, wherein Nintedanib esylate is present in an amount ranging from 30-60% w/w of the total suspension composition.

10. The soft gelatin capsule as claimed in claim 2, which further comprises medium chain triglyceride present in an amount ranging from 35-65% w/w of the total suspension composition.

11. The soft gelatin capsule as claimed in claim 2, which further comprises lecithin present in an amount ranging from 0.1-5% w/w of the total suspension composition.

12. The soft gelatin capsule comprises a capsule shell and a capsule composition, wherein the capsule composition comprises a composition as claimed in claim 2.
